(19) United States
(12) Patent Application Publication
(10) Pub. No.: US 2010/0130452 A1
(43) Pub. Date: May 27, 2010

(54) Use of Doxycycline in the Treatment of Idiopathic Pulmonary Fibrosis

(75) Inventor: Parthasarathi Bhattacharyya, West Bengal (IN)

Correspondence Address: THE WEBB LAW FIRM, P.C., 700 KOPPERS BUILDING, 436 SEVENTH AVENUE, PITTSBURGH, PA, 15219, US

(73) Assignee: NATIONAL RESEARCH DEVELOPMENT CORPORATION, New Delhi (IN)

(21) Appl. No.: 12/528,225

(22) PCT Filed: Feb. 25, 2008
(86) PCT No.: PCT/IN08/00108

§ 371 Date: Jan. 26, 2010

(30) Foreign Application Priority Data
Feb. 26, 2007 (IN) 278/KOL/2007

(51) Int. Cl. A61K 31/65; A61P 11/00

(52) U.S. Cl. 514/154; 514/152

(57) ABSTRACT

Use of doxycycline, a non specific matrix metalloproteinase inhibitor, for treatment of idiopathic pulmonary fibrosis and a pharmaceutical composition comprising doxycycline or its congener in combination with another compound selected from the drug listed for treatment of idiopathic pulmonary fibrosis in a standard dose.

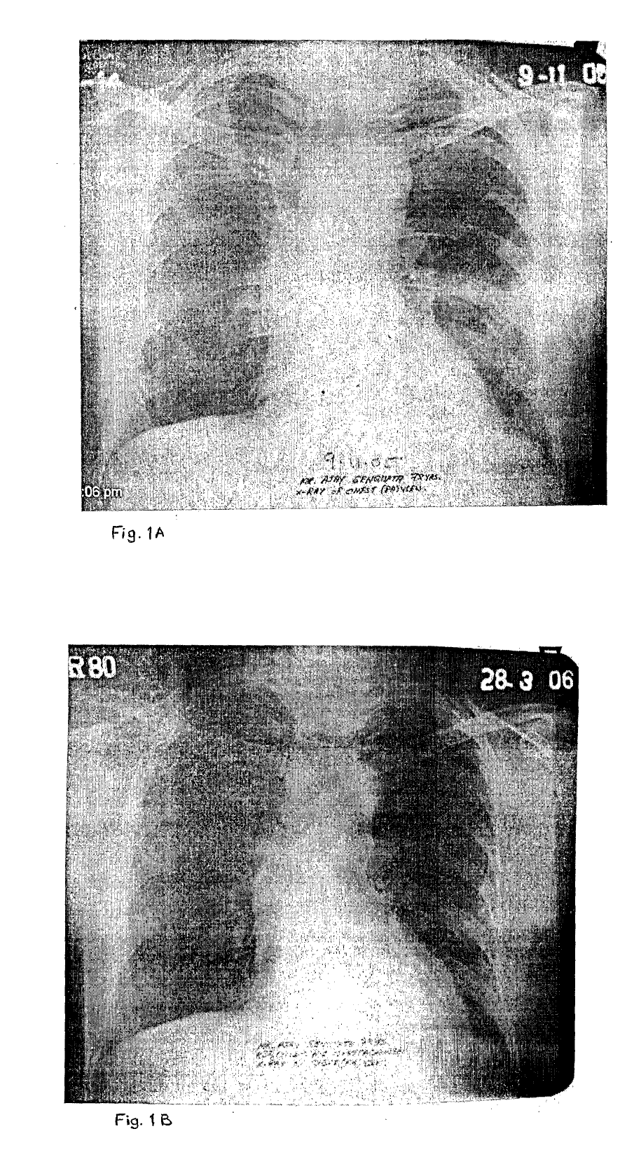

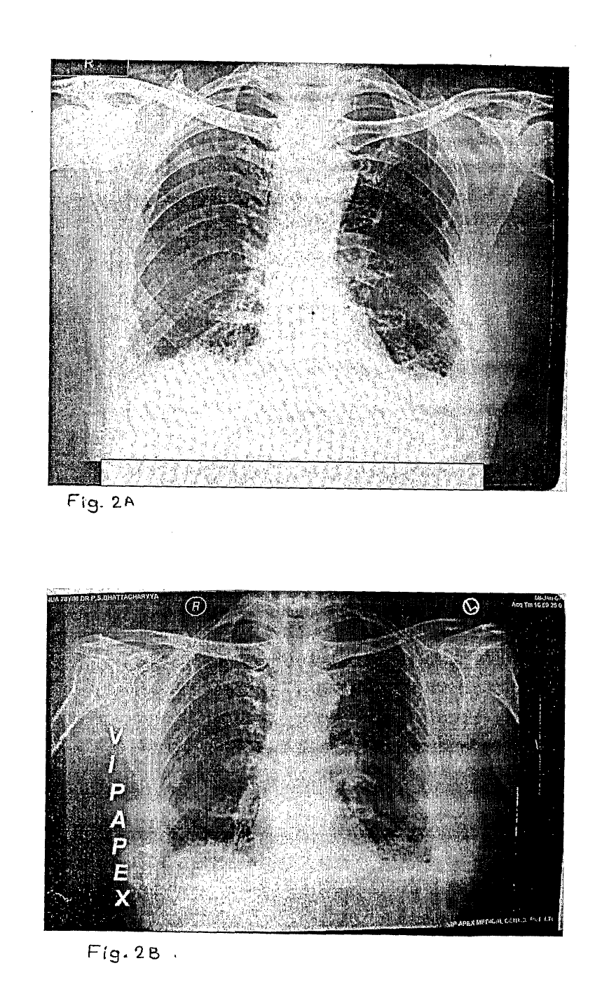

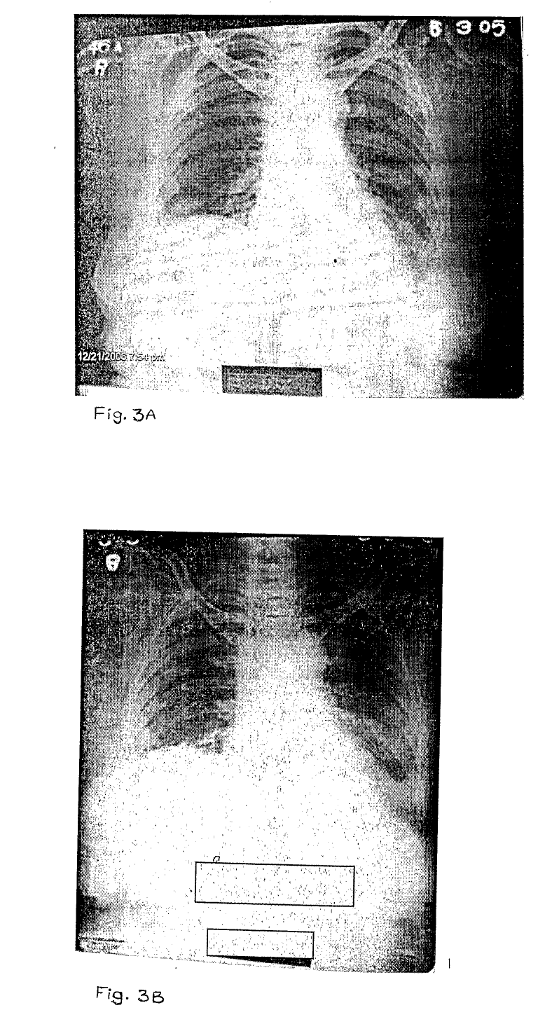

This invention relates to a chemical medicine for the treatment of Idiopathic pulmonary fibrosis.

### BACKGROUND OF THE INVENTION

Idiopathic pulmonary fibrosis (IPF) is a problem of progressive inflammation and fibrosis of lungs parenchyma without any apparent etiology. It can lead to significant morbidity and premature mortality. The present treatment of IPF is limited to the use of corticosteroid and immunosuppressive agents with the outcome being variable and the five year survival being 30 to 50 after the diagnosis being made.

The response to the conventional treatment of IPF with steroid and immunosuppressive agents is partial with no effect on honeycombing though the ground glass opacity is found to decrease in response to steroid. Current evidence does not support their routine use and longer trials of prednisolone and cytotoxic agents remain controversial since they have significant toxicities.

Antifibrotic therapy has been attempted with agents like colchicines and D-penicillamine that did not show any significant benefit. Pirfinidone, that blocks fibrogenic growth factor, extra cellular matrix secretion and fibroblast proliferation in vitro was reported well tolerated in ill patients in a small trial and is under further investigation. Some other agents as Relaxin, Suramin, Endothelin1 are also under investigation. Interferon gamma-1b (INFγ-1 b), a down regulator to TGFβ, has been found to increase lung volume, gas exchange and symptoms in an one year of study but a recent double blind controlled trial has ruled out its efficacy in IPF. Hence, because of the limitations of the medical therapy lung transplantation has emerged as an option for some patients. An effective and safe drug for IPF is looked out for internationally.

The prevailing pathophysiological concept of IPF has been recently shifting from inflammation induced fibrosis to a disease of fibroblast proliferation and dysregulated fibrogenesissis. Abnormal lung remodeling ensues from accumulation of extracellular matrix (ECM) following epithelial injury. There is dysregulated activity of matrix metalloproteinases (MMPs), a group of enzyme that can be related to release of fibroblast growth factor important for continuous stimulation of fibrogenesis in IPF. An imbalance in matrix metalloproteinases and tissue inhibitors of matrix metalloproteinases (TIMPs), delayed or absent re-epithelialization, and diminished vascularity are noted in IPF. The fibrogenesis may evolve from enhanced degradation of basement-membrane matrix and reduced production of TIMPs (tissue inhibitors of metalloproteinases). Hence, prevention of the MMP activity could be therapeutically beneficial for the patients of idiopathic lung fibrosis. In addition to this, there is fibroblast/myofibroblast migration and proliferation to sites of injury, decreased myofibroblast apoptosis, and increased activity of and response to fibrogenic cytokines such as transforming growth factor-β1 (TGF-β1), tumor necrosis factor-α, platelet-derived growth factor, and insulin-like growth factor at the fibroblastic foci that are characteristic of IPF. Evidence suggests that in the fibroblastic foci of IPF, there is less inflammation and absence of apoptosis allowing continued proliferation of myofibroblasts.

### OBJECTS OF THE INVENTION

An object of this invention is to propose a chemical medicine for the treatment of Idiopathic Pulmonary Fibrosis.

Another object of this invention is to propose a chemical medicine which has no significant side effects even on prolonged use of the same.

### DESCRIPTION OF THE INVENTION

According to this invention there is provided a use of doxycycline, a non-specific matrix-metalloproteinase inhibitor, for treatment of Idiopathic pulmonary fibrosis, and the dose for the adult is 100 mg every 12 hours during the first 24 hours followed by 100 mg once or twice a day depending on the body weight of the patient.

IPF patients respond very well to matrix metalloproteinase inhibitor doxycycline, because of its ability to interfere with matrix metalloproteinase (MMP) activity. Incidentally doxycycline has long been in use in clinical medicine without much side effects and long term use of doxycycline is also documented well in some conditions. Recently, there has been a report of successful treatment of pulmonary capillary hemangiomatosis with atypical endotheliomatosis refractory to steroid and alpha interferon by using doxycycline for over 18 months. In fact, tetracycline, doxycycline, and chemically modified tetracycline all can decrease MMP production and activity. Doxycycline is the only MMP inhibitor reportedly approved by the FDA for periodontal disease.

### Dosage of the Drug:

Doxycycline is a commonly used antibiotic. The adult dose is 100 mg every 12 hours during the first 24 hours followed by 100 mg once a day or twice daily according to the severity of the infection being present and the doctors' discretion. In situations where long term treatment is necessary, the dose mostly used is 100 mg twice daily. The dose used for treating a non infective condition named Pulmonary Capillary Hemangiomatosis with atypical Endotheliomatosis on a long term was also the same as 100 mg twice daily. The patient who received long term Doxycycline for the treatment of probable IPF by us was also treated with the dose of 100 mg twice a day. The same dose has been used in another case series where a follow up result after a mean duration of 306 days is analyzed. In all the examples the drug has been well tolerated. Hence, we propose the dose of 100 mg twice daily for adults of average weight for treatment of IPF on a long term basis.

However, the final and the best dose can only be determined after using the drug in different dosages in a proper clinical trial.

### Effect on the Patient:

The proposed mechanism of Doxycycline for treatment of Idiopathic pulmonary fibrosis (IPF) has been totally different from that applicable for its antimicrobial effect.

Doxycycline is a non specific matrix metallo-proteinase (MMPs) inhibitor. MMPs are a class of Zinc endopeptidase and they play an important part in modulation of fibrogenesis following epithelial injuries in cases of IPF. An imbalance with its tissue inhibitor in favour of MMPs leads to release of several growth factors including insulin-like growth factors (IGFs), transforming growth factors-β (TGF-β₁) and tumor necrosis factor α (TNF-α). They/these substances are known to enhance fibrosis. Indeed, a synthetic inhibitor of MMP, Batimastat has been shown to significantly reduce bleomycin induced lung fibrosis (in rats) that points to the importance of MMPs in the development of the fibrotic disease of the lungs. Since doxycycline has been available in the market over 30 years without much side effects and with experience of long term use and its MMPs inhibition is well known, the drug imparts a favorable effect on the patients of IPF

### Mode of Administration of the Drug:

The drug is administered orally preferably after 30 minutes to one hour of food to prevent disturbance in absorption. The duration of treatment with doxycycline depends on the indication concerned and it can be varying from a few days to a few years. For most infections, doxycycline is taken once or twice daily for 7 to 14 days. Infection with Mycoplasma pneumonae is treated for a duration of 10-14 days. Doxycycline has been recommended for use as a first line drug for brucellosis with streptomycin or rifampicin for 6 weeks. Field trials in 2005 showed that Doxycycline almost completely eliminates blood-borne filaria when given for an 8 week course.

Periodontal disease is a condition where doxycycline has been recommended for longer duration. In clinical studies, doxycycline hyclate (Periostat) was shown to be safe when taken for as long as 12 months and typically, the drug is taken for periods of no less than 3 months for periodontal disease. Prolonged therapy with doxy has been recorded well in toleration for over months. Doxycycline can be used as a substitute in penicillin allergic patients. In Q fever endocarditis doxycycline was given for a mean duration of 55 months (median 60 months) in combination with ofloxacin and for a mean duration of 31 months (median 26 months) with hydroxychloroquine where the patients treated were declared cured. Similar experience of Q-fever endocarditis has been described in a patient with biological prosthetic aortic valve and aortic homograft; the patient was successfully treated with doxycycline and chloroquine for 2 years. Doxycycline is licensed for up to two years or more in the treatment of acne in the same dose as is used for malaria prevention. The ACMP (Advisory Committee for Malaria Prevention of UK) have concluded that there is no evidence of harm in long-term use of doxycycline and it may be taken safely for periods of at least up to two years. A case of Pulmonary Capillary Hemangiomatosis with atypical endotheliomatosis was treated for 2 yrs without any problem. Recently, doxycycline has been reported to have a biological function apart from its antimicrobial function. Doxycycline is known to inhibit the release of reactive oxygen species, while also inducing apoptosis, decreasing neutrophil chemotaxis and inhibiting matrix metalloproteinases. One patient of suspected IPF has been treated by us for over seven months with 100 mg twice daily dose of doxycycline with no intolerance or adverse reaction. Also in a small case series prepared by us and ready for publication, it has been shown that the use of doxycycline for a mean period of 306 days in cases of suspected IPF patients was well tolerated.

Hence, we wish to recommend a dose of 100 mg BID (twice daily) for usual adult patients of IPF. However, trials with different dosages are needed to be conducted for finding out the best suitable dose of MMPs inhibition for IPF patients.

The invention will now be explained in details with the help of following test results and illustrate in the accompanying drawings.

### Example 1

### Response to Treatment with Only Doxycycline

### Case 1

AS, 73 years, male
HRCT suggestive of Idiopathic Pulmonary Fibrosis
Patient now in doxycycline (100 mg twice a day) for over six months

Improvement has been observed in:

a) Clinical symptomatology
b) Walking distance
c) Radiological parameters (x-rays enclosed)

### Case 2:

SJ, 78 years, Male
HRCT suggestive of Idiopathic Pulmonary Fibrosis and the bronchioalveolar lavage did not suggest any alternative diagnosis
Patient is presently on doxycycline (100 mg twice a day) for over 3 months

Improvement has been observed in:

a) Clinical symptomatology
b)6 Minute Walking distance (report enclosed)
c) Radiological parameters (x-rays enclosed)

### d) Saint George's Respiratory Questionnaire

e) Flow Volume Capacity (FVC) (report enclosed)
chest x-rays:
functional parameters:

| Date | 01 Oct. 2007 | 09 Jan. 2008 |
|---|---|---|
| FVC | 1.66 L | 1.92 L |
| 6 minute | 980 ft | 1155 ft |
| Walking distance | | |

### Case 3:

RRC, 84 years female
HRCT suggestive of Idiopathic Pulmonary Fibrosis
Patient has been on doxycycline (100 mg twice a day) for over 10 months

Improvement observed in

a) Clinical symptomatology
b) Radiological parameters (x-rays enclosed)

### c) Flow Volume Capacity (FVC)

d) Walking distance

### Case 4:

SM, 66 years male
HRCT suggestive of Idiopathic Pulmonary Fibrosis
Patient was evaluated after receiving Doxycycline (100 mg twice a day) for over 7 months

Improvement has been observed in

a) Clinical symptomatology
b) Radiological parameters
c) DLCO (diffusion capacity) and FEV1(forced expiratory volume in the first one second): results enclosed below
d) Walking distance

| DATE | 10 Jan. 2004 | 20 Aug. 2004 |
|---|---|---|
| FVC | 2.12 | 2.45 |
| FEV1 | 1.15 | 1.51 |
| TLCO(%) | 49.4 | 58.9 |
| TLCO/VA | 102.6 | 127.9 |
| VA | 3.76 | 3.59 |

### Example

### Treatment with Doxycycline+Azathioprine (Azoran), a Drug Presently Used for Treatment of Idiopathic Pulmonary Fibrosis (IPF)

### Case I

AS (55 years old female)
HRCT suggestive of Idiopathic Pulmonary Fibrosis
The Patient has been receiving Doxycycline (100 mg twice a day)+Azoran (1 mg per kg of body weight) for over 24 months

Improvement has been observed in

a) Clinical symptomatology
b) Walking distance
c) Radiological parameters (x-rays enclosed)

1-5. (canceled)

6. A method of treating idiopathic pulmonary fibrosis comprising:
utilizing a non specific matrix metalloproteinase inhibitor, wherein the non specific matrix metalloproteinase is doxycycline

7. The method of treating idiopathic pulmonary fibrosis as claimed in claim 6, wherein a dose of the doxycycline utilized for an adult is 100 mg every 12 hours during the first 24 hours followed by utilizing 100 mg once or twice a day.

8. The method of treating idiopathic pulmonary fibrosis as claimed in claim 6, wherein a dose of the dioxycycline utilized for a long term treatment is 100 mg once or twice daily.

9. The method of treating idiopathic pulmonary fibrosis as claimed in claim 8, wherein the dose for long term treatment is combined with standard therapy of idiopathic pulmonary fibrosis.

10. A pharmaceutical composition comprising a first compound and a second compound, wherein the first compound is selected from the group consisting of tetracycline, doxycycline, and chemically modified tetracycline and the second compound is selected from the group consisting of an immunosuppressive agent; a steroid; and an antibiotic.

11. The pharmaceutical composition of claim 10, wherein the first compound is doxycycline.

12. The pharmaceutical compound of claim 10, wherein the second compound is an immunosuppressive agent.

13. The pharmaceutical compound of claim 12, wherein the immunosuppressive agent is azathioprine.
